(19) United States
(12) Patent Application Publication
(10) Pub. No.: US 2024/0408120 A1
(43) Pub. Date: Dec. 12, 2024

(54) METHODS FOR INCREASING FAT ABSORPTION

(71) Applicant: STRUCTURED LIPID NUTRITION, LLC, Malvern Station, PA (US)

(72) Inventors: Virginia A. STALLINGS, Merion Station, PA (US); James P. O'CONNELL, JR., Merion Station, PA (US)

(73) Assignee: STRUCTURED LIPID NUTRITION, LLC, Merion Station, PA (US)

(21) Appl. No.: 18/700,437

(22) PCT Filed: Oct. 12, 2022
(86) PCT No.: PCT/US2022/046365

§ 371 Date: Apr. 11, 2024

Related U.S. Application Data
(60) Provisional application No. 63/254,786, filed on Oct. 12, 2021.

(51) Int. Cl. A61K 31/685; A61K 9/00; A61K 9/10; A61K 45/06; A61P 3/02

(52) U.S. Cl. CPC A61K 31/685; A61K 9/0053; A61K 9/10; A61K 45/06; A61P 3/02

(57) ABSTRACT

Provided herein are methods of increasing absorption of dietary fat and fat-soluble nutrients in individuals using a matrix comprising lysophosphatidylcholine (LPC).

### RELATED APPLICATION

This application claims priority to and the benefit of U.S. Application Ser. No. 63/254,786, filed Oct. 12, 2021, the content of which is incorporated herein by reference in its entirety.

### BACKGROUND

Fat malabsorption presents a significant challenge to the overall health and growth of individuals with poor nutritional uptake, such as those with exocrine pancreatic insufficiency (EPI) or bile acid deficiency. Although pancreatic enzyme replacement in EPI diseases and a high calorie diet can improve fat absorption in some affected individuals, these approaches are not always sufficient to restore fat absorption to normal levels to support optimal weight status in adults and healthy growth patterns in children.

### SUMMARY

The present disclosure provides methods of increasing intestinal absorption of dietary fat calories, essential fatty acids, and fat-soluble vitamins in individuals using a matrix comprising lysophosphatidylcholine (LPC), which may be in the form of micelles or micelle-like structures. When administered to an individual, LPC and/or LPC micelles or micelle-like structures enhance fat absorption in the digestive tract independently of the function of lipase enzymes that are typically secreted from the pancreas and bile acids that are typically secreted from the liver of healthy individuals. In some embodiments, the LPC and/or LPC micelles or micelle-like structures can be formulated as a lipid matrix that may be combined (mixed) with a food or beverage in an individual's diet to improve fat absorption from these sources of nutrition. In some embodiments, a formulation of LPC and/or LPC micelles or micelle-like structures may be used to improve the nutrition and/or growth of an individual having a particular disorder associated with impaired fat absorption, such as a disease associated with EPI, a disease associated with bile acid deficiency, or another disease.

In one aspect, the present provides a method of increasing dietary fat absorption comprising administering an effective amount of a matrix comprising LPC in combination with a food or beverage to a subject having impaired dietary fat absorption.

In some embodiments, dietary fat absorption in the subject is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

In some embodiments, the subject absorbs dietary fat from the matrix comprising LPC. In some embodiments, the subject absorbs dietary fat from the food or beverage. In some embodiments, the subject absorbs dietary fat from the food or beverage and not from the fat provided by the matrix comprising LPC. In some embodiments, the subject absorbs dietary fat from the food or beverage through recycling of LPC in the matrix from the circulatory system of the subject to the digestive tract.

In some embodiments, the administration increases the absorption of monounsaturated, polyunsaturated, and/or saturated fatty acids in the subject. In some embodiments, the fatty acids comprise one or more of the following: linoleic acid, α-linolenic acid, γ-linolenic acid, pentadecanoic acid, arachidonic acid, eicosapentaenoic acid, docosapentaenoic acid, and docosahexaenoic acid. In some embodiments, the administration increases the absorption of essential fatty acids in the subject. In some embodiments, the essential fatty acids comprise one or more of linoleic acid, α-linolenic acid, and pentadecanoic acid.

In some embodiments, dietary fat absorption in the subject is increased independently of lipase activity in the subject. In some embodiments, dietary fat absorption in the subject is increased independently of bile acids in the subject. In some embodiments, the dietary fat absorption improvement is higher in subjects with more severe fat malabsorption.

In some embodiments, the administration increases the absorption of fat-soluble vitamins and/or antioxidants in the subject. In some embodiments, the fat-soluble vitamins comprise one or more of vitamin A, vitamin D, vitamin E, and vitamin K.

In some embodiments, the subject is a human that is suspected of having or is known to have a disorder associated with impaired fat absorption. In some embodiments, the subject is a human neonate, a human infant, a human child, a human adult, or an elderly human. In some embodiments, the subject is an elderly human with frailty syndrome.

In some embodiments, the subject is a non-human animal, a domestic animal, or a companion animal that is suspected of having or is known to have a disorder associated with impaired fat absorption (e.g., malabsorption).

In some embodiments, the subject is suspected of having or is known to have a disease associated with exocrine pancreatic insufficiency (EPI). In some embodiments, the subject is suspected of having or is known to have cystic fibrosis, chronic pancreatitis, pancreatic cancer, type 1 diabetes mellitus, type 2 diabetes mellitus, type 3c diabetes mellitus, protein-calorie malnutrition, age-related lipase insufficiency, Schwachman-Diamond syndrome, Johanson-Blizzard syndrome, Zollinger-Ellison syndrome, and lipase/co-lipase enzyme deficiency.

In some embodiments, the subject is suspected of having or is known to have a disease associated with bile acid insufficiency. In some embodiments, the subject is suspected of having or is known to have cholestasis, cirrhosis, biliary atresia, Wilson disease, parental nutrition-associated liver disease, infectious hepatitis, hepatic steatosis with obesity, bile acid metabolism disorders, bilirubin metabolism disorders, end stage liver disease, and bile acid insufficiency associated with liver transplant.

In some embodiments, the subject is suspected of having or is known to have malabsorption syndrome, alcoholic pancreatitis, pancreatic duct obstruction, hereditary hemochromatosis, liver disease, liver cancer, bile duct obstruction, bile acid depletion, abetalipoproteinemia, cholecystitis, cholelithiasis, chronic gallbladder disease, gallbladder cancer, inflammatory bowel disease, irritable bowel syndrome, intestinal obstruction, intestinal pseudo-obstruction, intestinal adhesion, Crohn's disease, Celiac disease, ulcerative colitis, infectious colitis, ischemic colitis, radiation colitis, chronic constipation, chronic diarrhea, dysentery, peptic ulcer disease, gastritis, gastroenteritis, gastroesophageal reflux disease, acid reflux, functional dyspepsia, non-ulcer dyspepsia, gastroparesis, Barrett's esophagus, achalasia, non-achalasia esophageal motility disorders, anorexia nervosa, bulimia nervosa, orthorexia nervosa, avoidant restrictive food intake disorder, diabulimia, other specified feeding and eating disorders, intestinal cancer, colorectal cancer, appendicitis, diverticulitis, lactose intolerance, short bowel syndrome, abdominal adhesions, Whipple's disease, Pearson marrow-pancreas syndrome, Alagille syndrome, and Addison's anemia.

In some embodiments, administration of the matrix comprising LPC to the subject is followed by normalization of growth in the subject. In some embodiments, administration of the matrix comprising LPC to the subject improves weight status and/or management of a disorder associated with impaired fat absorption in the subject.

In some embodiments, the matrix comprising LPC is administered prior to, with, or following administration of a treatment for EPI, a treatment for the specific disease associated with EPI (e.g., cystic fibrosis), or a combination thereof. In some embodiments, the treatment for EPI comprises pancreatic enzyme replacement therapy (PERT). In some embodiments, the treatment for the specific disease associated with EPI is a treatment for cystic fibrosis that comprises a cystic fibrosis transmembrane conductance regulator (CFTR) corrector, a CFTR potentiator, or a combination thereof.

In some embodiments, the subject is a healthy non-human animal, a healthy domestic animal, or a healthy companion animal in which the matrix comprising LPC is administered for the purpose of increasing growth or weight gain.

In some embodiments, administration of the matrix comprising LPC increases the level of one or more nutritional status biomarkers in the subject, wherein the nutritional status biomarkers are selected from at least one fat for calories, at least one essential fatty acid, and/or least one fat soluble vitamin. In some embodiments, a fat for calories is a saturated fatty acid, a monounsaturated fatty acid, or a polyunsaturated fatty acid. In some embodiments, an essential fatty acid is linolenic acid, α-linolenic acid, or pentadecanoic acid. In some embodiments, a fat-soluble vitamin is vitamin A, vitamin D, vitamin E, or vitamin K.

In some embodiments, the matrix comprising LPC is at least 1% LPC by weight.

In some embodiments, the matrix comprising LPC is in the form of a dry powder, a paste, or a liquid. In some embodiments, the matrix comprising LPC is in the form of a dry powder. In some embodiments, the matrix comprising LPC is in the form of a dry powder that is mixed with a food or beverage.

In some embodiments, 0.5-50 g of the matrix comprising LPC are administered to the subject. In some embodiments, the matrix comprising LPC is administered to the subject at least once per day and/or at least once per week. In some embodiments, the matrix comprising LPC is administered at least twice per day.

In another aspect, the present disclosure provides a method of increasing fat absorption from the digestive tract comprising administering an effective amount of a matrix comprising LPC to a subject having impaired dietary fat absorption, whereby absorption of fat present in the digestive tract is increased.

In some embodiments, the absorption of fat present in the digestive tract is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

In some embodiments, the fat absorbed in the digestive tract of the subject is that which is comprised by the matrix comprising LPC. In some embodiments, the fat absorbed in the digestive tract of the subject is not solely that which is comprised by the matrix comprising LPC. In some embodiments, the fat absorbed in the digestive tract of the subject is absorbed through recycling of LPC in the matrix from the circulatory system to the digestive tract of the subject.

In another aspect, the present disclosure provides a method of increasing the concentration of fatty acids in plasma of a subject having impaired dietary fat absorption, comprising administering to the subject a source of fat and an effective amount of a matrix comprising lysophosphatidylcholine (LPC), whereby the subject absorbs fatty acids from the source of fat. In some embodiments, the source of fat is a food or beverage.

In some embodiments, the fatty acids comprise monounsaturated, polyunsaturated, and/or saturated fatty acids. In some embodiments, the fatty acids comprise one or more of linoleic acid, α-linolenic acid, γ-linolenic acid, pentadecanoic acid, arachidonic acid, eicosapentaenoic acid, docosapentaenoic acid, and docosahexaenoic acid.

In some embodiments, the concentration of fatty acids in plasma of the subject is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

In some embodiments, fatty acid absorption in the subject is increased independently of lipase activity and/or bile acid concentration in the intestines in the subject. The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### DETAILED DESCRIPTION OF CERTAIN EMBODIMENTS

Digestive disorders are highly prevalent, affecting an estimated 60 to 70 million people in the United States annually according to the National Institutes of Health (NIH) National Institute of Diabetes and Digestive and Kidney Diseases (NIDDK), as well as many millions more worldwide. Common symptoms of digestive disorders include gastrointestinal pain, indigestion, bloating, and fatty stools (steatorrhea). Numerous digestive disorders interfere with the function of digestive enzymes released by the pancreas and with the function of bile acids from the liver to extract nutrients from food, as well as the uptake of these nutrients by cells lining the small intestine. Many digestive disorders particularly interfere with the digestion and absorption of fats for calories and various fat-soluble nutrients, including essential fatty acids and fat-soluble vitamins. When digestive disorders persist over a prolonged period of time, they may cause weight loss and/or the inability to gain weight as a result of fat malabsorption. Of particular concern are chronic digestive disorders that can impede fat absorption for months or years, such as those caused by various surgical interventions, enteral infections, eating disorders, and hereditary diseases. Some chronic digestive disorders, such as exocrine pancreatic insufficiency (EPI), may be partially treated by administering pancreatic enzyme replacement therapy (PERT) and supplementing an individual's diet to be higher in fat. However, neither of these approaches fully restore normal levels of fat absorption for all digestive disorders in all patients, nor do these approaches consistently resolve each and every symptom of fat malabsorption. Meta-analyses of PERT in patients with chronic pancreatitis has shown that while PERT generally improves fat absorption, patient responses are highly variable (see, for example, de la Iglesia-Garcia, et al., “Efficacy of pancreatic enzyme replacement therapy in chronic pancreatitis: systematic review and meta-analysis” Gut 2017; 66:1354-1355). While PERT also tends to improve fat absorption in patients with cystic fibrosis and other EPI diseases, it fails to fully restore fat absorption to levels observed in healthy individuals and some patients continue to have symptoms of fat malabsorption, such as gastrointestinal pain and diarrhea (see, for example, Woestenenk, et al., “Pancreatic enzyme replacement therapy and coefficient of fat absorption in children and adolescents with cystic fibrosis”, J. Pediatr. Gastroenterol. Nutr. 2015; 61(3):355-360). Approximately 10% of patients with CF in the US require treatment with a feeding tube to provide additional calories even with optimized PERT. However, an increased consumption of dietary fat can also exacerbate symptoms in many patients, as this may concurrently increase the total quantity of dietary fat that is malabsorbed. In spite of the demonstrated need for therapies that consistently and robustly improve digestive health, the range of treatments available for addressing fat malabsorption have been limited thus far.

The following disclosure contemplates medical foods based on a lysophosphatidylcholine (LPC) matrix for use in improving the absorption of nutrients in the digestive tract, particularly fat. As used herein, the term “medical food” refers to any formulation that is suitable for enteral administration to (oral consumption or enteral feeding tube) a subject in order to provide a health benefit to the subject. A medical food may provide a benefit to the subject to whom it is administered when administered alone or when administered in administered in the presence of additional food or beverage. A medical food may provide an increased health benefit to the subject to whom it is administered when administered in the presence of additional food or beverage, compared to administration of the medical food alone. A medical food may be for use in the treatment of a particular disease or disorder. A medical food may be administered alone or with additional food or beverage to treat a disease or disorder, or may be combined with another treatment for the disease or disorder (e.g., enteral or non-enteral treatments), whether administered concurrently or sequentially, or administered by the same or different routes. A medical food contemplated herein may, for example, provide an enhancement in the absorption of one or more macronutrients (e.g., fat) and/or micronutrients (e.g., essential fatty acids, vitamins) in the subject.

The medical foods described herein and uses thereof include a matrix comprising LPC, a lipid biomolecule that enhances fat absorption in the digestive tract. In some patients, such a matrix may enhance fat absorption independently of the activity of endogenous digestive enzymes, such as pancreatic lipase and bile acid concentration. In some patients, such a matrix may enhance fat absorption in conjunction with the activity of endogenous digestive enzymes, such as pancreatic lipase. As used herein, the terms “matrix”, “lipid matrix”, and “structured lipid” are used interchangeably. Also described herein are formulations comprising an LPC matrix, which comprises LPC, monoglycerides, and fatty acids (e.g., in a 1:4:2 molar ratio). Also described herein are methods for administering such matrices or formulations to a subject for the purpose of improving fat absorption. The formulations described herein are useful for treating fat malabsorption due to their improved palatability, flexible dosage, improved nutrient absorption capabilities, and suitability for a broad range of digestive disorders.

### Lysophosphatidylcholine and LPC Micelle-Like Structures

The present disclosure provides a matrix comprising lysophosphatidylcholine (LPC), a water-soluble lipid which is alternatively referred to as lysoPC or lysolecithin. LPC is produced from the partial hydrolysis of phosphatidylcholine (PC), a class of phospholipids which contain a head group consisting of the essential nutrient choline and glycerol 3-phosphate (glycerophosphoric acid), the latter of which is in turn covalently linked to two fatty acid tail groups (e.g., unsaturated or saturated fatty acids). LPC is produced from the hydrolysis (removal) of either of the fatty acid tail groups of PC. LPC occurs in either of two isomeric subclasses: 1-LPC and 2-LPC, which are distinguished by the position of an acyl group within glycerol 3-phosphate of the head group. 2-LPC is produced from 1-LPC as a result of the migration (transfer) of the acyl group at the sn-1 (first carbon) position of the head group to the sn-2 (second carbon) position, as determined by the stereospecific numbering (sn) system for lipids that is well known within the art. Conversely, 1-LPC is produced from 2-LPC as a result of the acyl migration in the reverse direction. Endogenous LPC is synthesized from PC in a variety of tissues, such as the liver by the activity of phospholipase A₁ (PLA1; EC 3.1.1.32) or phospholipase A₂ (PLA2; EC 3.1.1.4), which catalyze the hydrolysis (removal) of fatty acids attached to the sn-1 or sn-2 acyl group, respectively, of various phospholipids, such as PC. LPC may also be synthesized as a by-product of reactions catalyzed by other enzymes, such as lecithin-cholesterol acyltransferase (LCAT; EC 2.3.1.43), which transfers a fatty acid from PC onto cholesterol. The rate of isomerization between 1-LPC and 2-LPC depends upon the acidity of the surrounding solvent, with lower rates occurring in acidic conditions. At equilibrium, LPC predominantly occurs as 1-LPC in acidic conditions, while the majority of endogenous LPC, including that which is bound by plasma albumin, is thought to occur as 2-LPC at physiological pH, e.g., pH 7, due to the rapid isomerization of 1-LPC to 2-LPC above about pH 6 (see, for example, Croset M, et al. “Characterization of plasma unsaturated lysophosphatidylcholines in human and rat.” Biochem J. 2000; 345 Pt 1:61-67). Further details relating to the metabolism and function (e.g., signaling function) of 1-LPC and 2-LPC may be found, for instance, in Law S H, et al. “An Updated Review of Lysophosphatidylcholine Metabolism in Human Diseases.” Int J Mol Sci. 2019; 20(5):1149. Unless otherwise specified, the term “LPC” as used herein should be understood to specify LPC comprising 1-LPC, 2-LPC, or a combination (mixture) thereof, such as, for example, a mixture of 1-LPC and 2-LPC where the overall rate of isomerization between these isoforms is at equilibrium.

The general structures of 1-LPC and 2-LPC are provided below in Formula (I) and Formula (II), respectively, where ‘R’ denotes any fatty acid group (e.g., any unsaturated or saturated fatty acid).

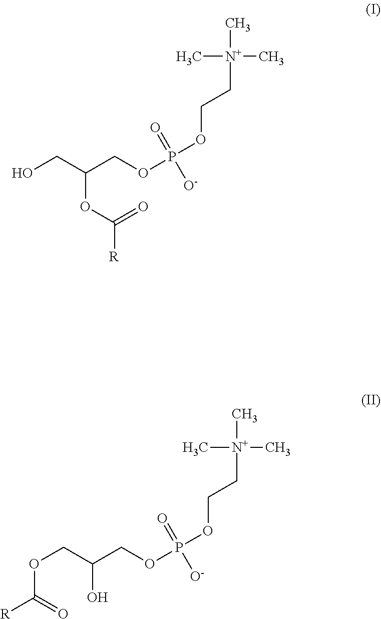

Without wishing to be bound by theory, dietary fats, e.g., triacylglycerols and diglycerides, are broken down into fatty acids and monoglycerides by endogenously expressed pancreatic lipase (EC 3.1.1.3) and colipase (CLPS) proteins. These digested fats subsequently interact with bile salts (bile acids) during digestion. Upon interacting with bile salts (e.g., cholic acid, glycocholic acid, taurocholic acid, deoxycholic acid, glycochenodeoxycholic acid, taurochenodeoxycholic acid), digested dietary fats assemble into micelles having a lipophilic core and hydrophilic exterior due to the amphipathic property of bile salts. These micelles are then absorbed by epithelial cells (e.g., mucosal cells) lining the intestine (e.g., in intestinal microvilli of the small intestine), where they are disassembled and incorporated into lipoprotein particles for secretion into the bloodstream for transport to tissues throughout the body. Further details relating to the function of pancreatic lipase during digestion may be found, for instance, in Sensoy I. “A review on the food digestion in the digestive tract and the used in vitro models”. Curr Res Food Sci. 2021 Apr. 14; 4:308-319.

Although bile salts facilitate the efficient uptake of digested dietary fats (e.g., fatty acids, monoglycerides) and certain other nutrients (e.g., fat-soluble vitamins), other, similarly amphipathic molecules may also assemble into micelles that can be absorbed through the intestinal lining, independent of the function of pancreatic lipase and bile salts. LPC is found in a wide range of foods as a component of cellular membranes and is among the most abundant phospholipids in the mucosal lining of the intestinal tract. Recent studies have also suggested a role for choline-containing phospholipids in facilitating the uptake and transport of dietary fat (see, e.g., Nakano T, et al. “Lysophosphatidylcholine for efficient intestinal lipid absorption and lipoprotein secretion in Caco-2 cells”. J Clin Biochem Nutr. 2009; 45(2):227-23; Li Z, et al. “Deficiency in lysophosphatidylcholine acyltransferase 3 reduces plasma levels of lipids by reducing lipid absorption in mice”. Gastroenterology. 2015 November; 149(6):1519-29; and Kabir I, et al. “Small Intestine but Not Liver Lysophosphatidylcholine Acyltransferase 3 (Lpcat3) Deficiency Has a Dominant Effect on Plasma Lipid Metabolism”. J Biol Chem. 2016 Apr. 1; 291(14):7651-60). Due to its presence in the digestive tract, LPC, which contains a lipophilic tail group and a positively charged choline-containing head group, may assemble into micelles that interact with digested dietary fats (e.g., free fatty acids, monoglycerides) and certain nutrients (e.g., fat-soluble vitamins) to facilitate absorption of these nutrients across the intestinal lining. A micelle comprising LPC that effectively facilitates absorption of dietary fats and other fat-soluble nutrients may be composed entirely of LPC or may be composed of LPC and additional lipophilic substances, such as dietary fats (e.g., fatty acids, monoglycerides).

In turn, a mechanism has been reported for the retrieval of LPC from lipoprotein particles circulating in the bloodstream, wherein LPC is returned to the gastrointestinal tract lumen through a paracellular route via tight junctions in gut mucosal cells of the intestinal epithelia (see, for example, Stremmel W, et al. “Phosphatidylcholine passes through lateral tight junctions for paracellular transport to the apical side of the polarized intestinal tumor cell-line CaCo2.” Biochim Biophys Acta. 2016; 1861(9 Pt A):1161-1169; and Stremmel W, et al. “The neglected biliary mucus and its phosphatidylcholine content: a putative player in pathogenesis of primary cholangitis-a narrative review article.” Ann Transl Med. 2021; 9(8):738). LPC transport by this paracellular route is facilitated by multidrug resistance protein 3 (MDR3; alternately referred to as ATP-binding cassette subfamily B member 4 (ABCB4)), a transporter protein (pump) expressed by mucosal cells lining the intestine and bile duct that expels a variety of circulating complex molecules into the intestinal lumen. As a result of MDR3 activity, LPC may be recycled to the intestinal tract lumen, thereby facilitating the continued absorption of dietary fats and fat-soluble nutrients from food and beverage. Moreover, exogenous LPC (e.g., LPC comprised by a medical food matrix) may be administered to subjects for the purpose of further facilitating fat absorption.

Compositions and uses for LPC contemplated herein may utilize LPC that is assembled into a structured lipid matrix. A matrix comprising LPC may be arranged in any of several possible phases (topologies) known in the art. A matrix comprising LPC may, for example, be arranged in a lamellar (sheet) phase, or a hexagonal or inverse hexagonal (micellar) phase. A matrix comprising LPC in a hexagonal phase or inverse hexagonal phase (e.g., a matrix comprising a LPC micelle) may comprise LPC micelles arranged with a lipophilic core and hydrophilic exterior (hexagonal phase), with a hydrophilic core and a lipophilic exterior (i.e., inverse hexagonal phase), or with a combination of arrangements thereof.

A matrix comprising LPC contemplated herein (e.g., a matrix comprising a LPC micelle), may comprise LPC and one or more additional lipophilic substances (e.g., triglycerides, triacylglycerols, diglycerides, monoglycerides, fatty acids, fat-soluble vitamins). A matrix comprising LPC may comprise one or more glycerides (e.g., a monoglyceride, a diglyceride, a triglyceride) and one or more fatty acids (e.g., oleic acid, linolenic acid) in addition to LPC. A matrix comprising LPC, one or more glycerides, and one or more fatty acids may comprise a molar ratio of LPC to the sum of glycerides and fatty acid that is between about 1:3 and about 1:12, preferably a molar ratio that is about 1:6. A matrix comprising LPC, one or more glycerides, and one or more fatty acids may, for example, comprise these components in a molar ratio of LPC:glyceride:fatty acid that is between about 1:4:2 and about 1:2:4. A matrix comprising LPC, one or more glycerides, and one or more fatty acids may, for example, have a molar ratio of LPC:glyceride:fatty acid that is about 1:4:2, about 1:3:2, about 1:3:3, about 1:2:3, or about 1:2:4.

In certain preferred embodiments, a matrix comprising LPC comprises LPC, monoglycerides, and fatty acids in a 1:4:2 molar ratio. Without wishing to be bound by theory, LPC comprised by the matrix supports absorption of dietary fats, while monoglycerides and fatty acids comprised by the matrix are directly absorbed once hydrolyzed in the intestinal lumen.

A matrix comprising LPC contemplated herein (e.g., a matrix comprising a LPC micelle), may comprise micelles composed essentially of LPC, micelles comprising LPC and one or more additional lipophilic substances (e.g., triglycerides, triacylglycerols, diglycerides, monoglycerides, fatty acids, fat-soluble vitamins), or a combination of micelles thereof. A matrix comprising LPC may comprise one or more solvents, such as water. A solvent may be located, for example, in the space between LPC sheets (e.g., in a matrix arranged in a lamellar phase), in the space between LPC micelles (e.g., in a matrix arranged in a hexagonal phase), or in the space within LPC micelles (e.g., in a matrix arranged in an inverse hexagonal phase).

### Edible Formulations Comprising LPC

Various formulations of matrices comprising LPC are also described. As used herein, the terms “formulation” and “composition” may be used interchangeably.

Previous formulations comprising LPC matrices include Lym-X-Sorb (also referred to as LXS), a formulation comprising LPC developed for enteral administration (oral consumption) as a nutritional supplement (see, for example, U.S. Pat. Nos. 4,874,795, 5,891,466, 5,707,873, and 6,426,069). Yesair and colleagues formulated the lipid matrix of Lym-X-Sorb to comprise LPC, monoglyceride, and fatty acids (e.g., in a 1:3:3 molar ratio) and observed it to be a highly viscous, non-Newtonian fluid. Lym-X-Sorb was found to be unpalatable, having a bitter taste, particularly when arranged in a lamellar or hexagonal phase, which was attributed to the externally facing choline head groups of LPC. The palatability of Lym-X-Sorb could be improved by adding about 8 moles of water per 1 mole of lipid matrix and heating to 50-60° C., which was suggested to reduce the bitterness of the matrix by rearranging it into an inverse hexagonal arrangement, thereby positioning the choline head groups of LPC on the inside of micelles (see, for example, U.S. Pat. Nos. 7,407,779, and 7,785,621). Yesair and colleagues further described the use of Lym-X-Sorb for supplementing the diet of subjects having nutritional disorders, such as wasting diseases, cancer, and cystic fibrosis (see, for example, Lepage G, et al. “Effect of an organized lipid matrix on lipid absorption and clinical outcomes in patients with cystic fibrosis.” J Pediatr. 2002; 141(2):178-85). Although palatability and mixing characteristics of Lym-X-Sorb were improved by modifying the formulation (then comprising LPC, triglycerides, essential fatty acids, sugar, and wheat flour (about 34% kcal from lipids, about 58% kcal from carbohydrates, about 6% kcal from protein); see, for example, Groleau V, et al. “Effect of a dietary intervention on growth and energy expenditure in children with cystic fibrosis.” J Cyst Fibros. 2014; 13(5):572-578), palatability continued to limit administration of the matrix to subjects, primarily due to a need to mask the taste and texture of the matrix (see, for example, Hommel K A, et al. “Adherence to Nutritional Supplementation in Cystic Fibrosis.” J Pediatr Nurs. 2019; 47:18-22). LPC matrices are effective for improving fatty acid absorption and growth in cystic fibrosis patients (see, for example, Stallings V A, et al. “Improved residual fat malabsorption and growth in children with cystic fibrosis treated with a novel oral structured lipid supplement: A randomized controlled trial.” PLoS One. 2020; 15(5):e0232685).

Unless specified otherwise, a matrix comprising LPC that is disclosed herein is to be formulated in a manner that is suitable for enteral administration to (oral consumption by) a subject (i.e., such formulations are edible). In some embodiments, a matrix comprising LPC is formulated as a medical food for use in improving fat absorption in a subject to which the formulation is administered.

In some embodiments, a matrix comprising LPC is formulated to comprise at least 0.5%, at least 1%, at least 2%, at least 3% at least 4%, at least 5%, at least 6%, at least 7%, at least 8%, at least 9% or at least 10% LPC by weight, but less than 20% LPC by weight. In some embodiments a matrix comprising LPC is formulated to comprise at least 2% but less than 5% LPC by weight. As used herein, the term “weight” is used synonymously with the term “mass.”

A matrix comprising LPC may be formulated to comprise additional pharmaceutically acceptable materials (ingredients). Such materials that may be added, for example, include one or more macronutrients (e.g., fats, carbohydrates), micronutrients (e.g., vitamins), isotonicizers (e.g., salts), emulsifiers, or palatability enhancers. Examples of macronutrients include fats, such as monoglycerides and fatty acids (e.g., oleic acid, linolenic acid), as well as carbohydrates, such as polysaccharides (e.g., starches, such as corn starch, tapioca starch, potato starch, and wheat starch), oligosaccharides (e.g., lactose, fructo-oligosaccharides), monosaccharides (e.g., glucose (dextrose), fructose, and galactose), and proteins (e.g., pea protein powder). Examples of isotonicizers include salts, such as calcium chloride, that control the osmolarity of the formulation (e.g., when administered to a subject). Examples of emulsifiers include soybean lecithin, as well as various other emulsifiers known in the art.

A matrix comprising LPC may be formulated to further comprise a flour produced by grinding a vegetable grain, seed, nut, bean, or tuber. Examples of suitable flours include tapioca flour, wheat flour, rice flour, corn flour, potato flour, oat flour, barley flour, rye flour, millet flour, soy flour, buckwheat flour, bulgur flour, sorghum flour, amaranth flour, quinoa flour, kamut flour, teff flour, flaxseed flour, peanut flour, almond flour, walnut flour, pecan flour, cashew flour, hazelnut flour, or coconut flour.

A matrix comprising LPC may be formulated to further comprise a flavorant (e.g., a flavor derived from a natural source, such as a fruit, nut, spice, or a synthetically produced equivalent), a sweetener (e.g., glucose (dextrose), fructose, sucrose, lactose, maltodextrin, invert sugar, sucralose, sodium saccharin, aspartame), a humectant (e.g., glycerin, hydrolyzed wheat, honey, corn syrup, sorbitol), a colorant (e.g., an edible dye), an anti-caking additive (e.g., tricalcium phosphate, sodium bicarbonate, powdered cellulose), a preservative (e.g., an antimicrobial agent, e.g., sodium benzoate, sodium nitrite, sodium sorbate, potassium sorbate, sulfur dioxide, propionic acid), or a salt (e.g., sodium chloride).

A matrix comprising LPC may be formulated as a solid (e.g., as a powder), as a semi-solid (e.g., as a paste), or as a liquid. A matrix comprising LPC may be formulated in such a way as to be directly suitable for administration to a subject, or may be combined as an ingredient with one or more additional ingredients to produce a formulation that is suitable for administration to a subject.

In some embodiments, a matrix comprising LPC is formulated as a dry, flowable powder. In embodiments where a matrix comprising LPC is formulated as a dry, flowable powder, the formulation is suitable to be combined (e.g., mixed) with another source of fat, such as a food or beverage, prior to administration to a subject. In some embodiments, a matrix comprising LPC is formulated such that it is miscible with liquids (e.g., the formulation rapidly dissolves in water).

In some embodiments, a matrix comprising LPC that is formulated as a dry, flowable powder is packaged in bulk or in an amount sufficient for a single dose (i.e., unit dose). In some embodiments, a matrix comprising LPC that is formulated as a dry, flowable powder is packaged in a pouch, bag, bottle, jar, cup, or other vessel similarly known in the art for the storage of edible compositions.

### Methods for Administering a Matrix Comprising LPC to Subjects

Also provided herein are methods for improving fat absorption in a subject (individual) in need thereof comprising administering to the subject an effective amount of a matrix comprising LPC or a formulation comprising such a matrix as described herein. In some embodiments, the subject in need is a subject having impaired dietary fat absorption. As used herein, the term “therapeutically effective amount” refers to an amount that is necessary and sufficient to have an intended therapeutic effect in a subject (e.g., an amount that is necessary and sufficient to improve fat absorption and improve weight and nutritional status (e.g., essential fatty acid levels, fat soluble vitamin levels) in a subject). In some embodiments, the effective amount of a matrix comprising LPC or a formulation thereof is enterally administered to the subject in need (i.e., is enterally consumed by the subject) by any means known in the art such as, for example, direct oral administration to the subject or administration via a feeding tube. In some embodiments, a formulation comprising the matrix comprising LPC is in the form of a medical food. In some embodiments, the matrix comprising LPC or a formulation thereof is administered to a subject alone. In some embodiments, the matrix comprising LPC or a formulation thereof is administered to a subject in combination with another source of fat (e.g., a food or beverage).

In embodiments where the matrix comprising LPC or a formulation thereof is administered in combination with another source of fat (e.g., a food or beverage), the matrix comprising LPC or formulation thereof may be administered concurrently with the other source of fat (e.g., as a mixture), or may be administered sequentially. In embodiments where a matrix comprising LPC or formulation thereof is administered concurrently with another source of fat (e.g., a food or beverage), the matrix comprising LPC or formulation thereof may be combined with the source of fat by any means readily known in the art such as, for example, mixing when the source of fat is a solid, semi-solid, or liquid composition. Example solid, semi-solid, or liquid sources of fats include fruits, vegetables, meats, cereals and grains, dairy, jellies, syrups, sauces, soups, shakes, smoothies, and juices. In some embodiments, the matrix comprising LPC or formulation thereof may be combined with the source of fat by cooking or baking together. Example cooked or baked sources of fat include cookies, wafers, bars, cakes, breads, cooked fruits, cooked vegetables, cooked meats, and cooked cereals and grains. In some embodiments, the matrix comprising LPC or formulation thereof may be combined with one or more other nutritional supplements such as, for example, a liquid nutritional supplement for weight gain or for maintaining electrolyte balance (e.g., a ready-to-drink liquid nutritional supplement).

In some embodiments, administration of a matrix comprising LPC or a formulation thereof to a subject increases (enhances) fat absorption in the subject by at least 10%, by at least 20%, by at least 30%, by at least 40%, by at least 50%, by at least 60%, by at least 70%, by at least 80%, by at least 90%, by at least 2-fold, by at least 3-fold, by at least 4-fold, by at least 5-fold, by at least 6-fold, by at least 7-fold, by at least 8-fold, by at least 9-fold, or by at least 10-fold. In some embodiments, fat absorption in the subject is increased in the digestive tract.

In some embodiments, a subject may absorb fat in the digestive tract that is comprised by LPC, LPC micelles or micelle-like structures, or a formulated matrix thereof. In such embodiments, administration of a matrix comprising LPC or a formulation thereof to a subject may improve fat absorption through the absorption of one or more dietary fats (e.g., one or more glycerides, one or more fatty acids) comprised by the administered matrix or formulation thereof.

In some embodiments, a subject may absorb fat in the digestive tract that is not solely that which is comprised by LPC, LPC micelles or micelle-like structures, or a formulated matrix thereof. In such embodiments, administration of a matrix comprising LPC or a formulation thereof to a subject may improve fat absorption from dietary food and beverages through recycling of micellar LPC from the circulatory tract to the digestive tract (e.g., via MDR3). In embodiments where administration of a matrix comprising LPC or a formulation thereof occurs in combination with a food or beverage, the subject may absorb dietary fat from the food or beverage. In such embodiments, a matrix comprising LPC may be formulated in such a way that absorption of dietary fat from the food or beverage is enhanced compared to administration of the LPC alone or in the absence of the LPC. In some embodiments, the subject may absorb dietary fat from the food or beverage and not from the matrix comprising LPC or a formulation thereof.

In some embodiments, administration of a matrix comprising LPC or a formulation thereof increases the absorption of fatty acids in the subject. In some embodiments, the fatty acids comprise monounsaturated, polyunsaturated, or saturated fatty acids, or a combination thereof. In some embodiments, the fatty acids comprise one or more of linoleic acid, α-linolenic acid, 7-linolenic acid, pentadecanoic acid, arachidonic acid, eicosapentaenoic acid, docosapentaenoic acid, and docosahexaenoic acid. In some embodiments, administration of the matrix comprising LPC or a formulation thereof increases (enhances) absorption of fatty acids in the subject by at least 10%, by at least 20%, by at least 30%, by at least 40%, by at least 50%, by at least 60%, by at least 70%, by at least 80%, by at least 90%, by at least 2-fold, by at least 3-fold, by at least 4-fold, by at least 5-fold, by at least 6-fold, by at least 7-fold, by at least 8-fold, by at least 9-fold, or by at least 10-fold. In some embodiments, administration of the matrix comprising LPC or a formulation thereof increases (enhances) the concentration of fatty acids in plasma of the subject by at least 10%, by at least 20%, by at least 30%, by at least 40%, by at least 50%, by at least 60%, by at least 70%, by at least 80%, by at least 90%, by at least 2-fold, by at least 3-fold, by at least 4-fold, by at least 5-fold, by at least 6-fold, by at least 7-fold, by at least 8-fold, by at least 9-fold, or by at least 10-fold. In some embodiments, fatty acid absorption in the subject is increased in the digestive tract.

In some embodiments, administration of the matrix comprising LPC or a formulation thereof increases the absorption of fat-soluble vitamins in the subject. In some embodiments, the fat-soluble vitamins comprise one or more of vitamin A (retinol, retinyl esters, beta carotene, and provitamin A carotenoids), vitamin D (calciferols), vitamin E (tocopherols and tocotrienols), and vitamin K (phylloquinone and menaquinones). In some embodiments, administration of the matrix comprising LPC or a formulation thereof increases (enhances) absorption of fat-soluble vitamins in the subject by at least 10%, by at least 20%, by at least 30%, by at least 40%, by at least 50%, by at least 60%, by at least 70%, by at least 80%, by at least 90%, by at least 2-fold, by at least 3-fold, by at least 4-fold, by at least 5-fold, by at least 6-fold, by at least 7-fold, by at least 8-fold, by at least 9-fold, or by at least 10-fold. In some embodiments, administration of the matrix comprising LPC or a formulation thereof increases (enhances) the concentration of fat-soluble vitamins in serum of the subject by at least 10%, by at least 20%, by at least 30%, by at least 40%, by at least 50%, by at least 60%, by at least 70%, by at least 80%, by at least 90%, by at least 2-fold, by at least 3-fold, by at least 4-fold, by at least 5-fold, by at least 6-fold, by at least 7-fold, by at least 8-fold, by at least 9-fold, or by at least 10-fold. In some embodiments, fat-soluble vitamin absorption in the subject is increased in the digestive tract.

In some embodiments, a subject to which a matrix comprising LPC or a formulation thereof is administered is a human, such as a human neonate, a human infant, a human child, a human adult, or an elderly human. In some embodiments, a subject to which a matrix comprising LPC or a formulation thereof is administered is a human neonate or human infant that is born prematurely, at, or after term. In some embodiments, a subject to which a matrix comprising LPC or a formulation thereof is administered is an elderly human who is frail (i.e., has frailty syndrome) and optionally one or more additional diseases or disorders, such as age-related lipase insufficiency.

In some embodiments, a subject to which a matrix comprising LPC or a formulation thereof is administered is a non-human animal. In some embodiments, the subject to which a matrix comprising LPC or a formulation thereof is administered is an immature non-human animal (e.g., a newborn (neonate or infant) or developing non-human animal) or a mature non-human animal (e.g., a fully grown (adult) or elderly non-human animal). In some embodiments, a subject to which a matrix comprising LPC or a formulation thereof is administered is a non-human animal that is born prematurely, at, or after term. In some embodiments, a subject to which a matrix comprising LPC or a formulation thereof is administered is an elderly non-human animal who is frail (i.e., has frailty syndrome) and optionally one or more additional diseases or disorders, such as age-related lipase insufficiency.

In some embodiments, a subject to which a matrix comprising LPC or a formulation thereof is administered is a production animal (a domesticated animal or an agricultural animal) such as, but not limited to, a mammal, a bird (avian), or a reptile. In some embodiments, a subject to which a matrix comprising LPC or a formulation thereof is administered is a production animal that is, for example, a bovine (Bos taurus), a sheep (Ovis aries), a goat (Capra aegagrus), a pig (Sus scrofa domesticus), a chicken (Gallus domesticus), a duck (Anas platyrhynchos domesticus), a goose (Anser anser domesticus), a turkey (Meleagris gallopavo domesticus), a horse (Equus ferus caballus), a donkey (Equus africanus asinus), a camel (Camelus dromedarius or Camelus bactrianus), a llama (Lama glama), an alpaca (Vicugnapacos), or a yak (Bos grunniens). In some embodiments, a production animal is a diseased animal (e.g., a production animal having impaired fat absorption) to which a matrix comprising LPC or a formulation thereof is administered for the purpose of increasing the rate of weight gain in said production animal. In some embodiments, a production animal is a healthy animal to which a matrix comprising LPC or a formulation thereof is administered for the purpose of increasing the rate of growth (size and/or weight, for example, in a young production animal) or rate of weight gain (for example, in a mature production animal) in said production animal.

In some embodiments, a subject to which a matrix comprising LPC or a formulation thereof is administered is a companion animal (a pet), a service animal, or a research animal such as, but not limited to, a mammal, a bird (avian), or a reptile. In some embodiments, a subject to which a matrix comprising LPC or a formulation thereof is administered is a companion animal, a service animal, or a research animal that is, for example, a dog (Canis lupusfamiliaris), a cat (Felis catus), a horse (Equusferus caballus), a mouse (Mus musculus), a rat (Rattus norvegicus), a rabbit (Oryctolagus cuniculus), or a guinea pig (Cavia porcellus). In some embodiments, a companion animal, a service animal, or a research animal is a diseased animal (e.g., a companion animal, service animal, or research animal having impaired fat absorption) to which a matrix comprising LPC or a formulation thereof is administered for the purpose of increasing the rate of weight gain in said companion animal, service animal, or research animal.

In some embodiments, a subject to which a matrix comprising LPC or a formulation thereof is administered is suspected of having or is known to have one or more disorders or diseases associated with impaired fat absorption. As used herein, the term “disease” and “disorder” are used interchangeably and may refer to either a transiently occurring (e.g., an acute medical event) or a chronically occurring disease or disorder. In some embodiments, a disorder associated with impaired fat absorption that occurs in a subject may be known or may not be known. In some embodiments, a subject is suspected of having or known to have a disorder associated with impaired fat absorption that is a disease associated with exocrine pancreatic insufficiency (EPI, i.e., levels of pancreatic lipase in the intestinal lumen that are insufficient to catalyze the digestion of dietary fats) that is selected from, but not limited to: cystic fibrosis, chronic pancreatitis, pancreatic cancer, diabetes mellitus (e.g., type 1, type 2, or type 3c (pancreatogenic) diabetes mellitus), protein-calorie malnutrition, age-related lipase insufficiency, Schwachman-Diamond syndrome, Johanson-Blizzard syndrome, Zollinger-Ellison syndrome, and lipase/co-lipase enzyme deficiency. In some embodiments, a subject is suspected of having or known to have a disorder associated with impaired fat absorption that is a disease associated with bile acid insufficiency (i.e., levels of bile acids (bile salts) in the intestinal lumen that are insufficient to promote the efficient formation and absorption of micelles containing dietary fats) that is selected from, but not limited to: cholestasis, cirrhosis, biliary atresia, Wilson disease, parental nutrition-associated liver disease, infectious hepatitis, hepatic steatosis with obesity, bile acid metabolism disorders, bilirubin metabolism disorders, end stage liver disease, or bile acid insufficiency associated with liver transplant. In further embodiments, a subject is suspected of having or known to have a disorder associated with impaired fat absorption that is selected from, but not limited to: malabsorption syndrome, alcoholic pancreatitis, pancreatic duct obstruction (biliary atresia), hereditary hemochromatosis, liver disease, liver cancer, bile duct obstruction, bile acid depletion, abetalipoproteinemia, cholecystitis, cholelithiasis (gallstones), chronic gallbladder disease, gallbladder cancer, inflammatory bowel disease, irritable bowel syndrome, intestinal obstruction, intestinal pseudo-obstruction, intestinal adhesion, Crohn's disease, Celiac disease, ulcerative colitis, infectious colitis, ischemic colitis, radiation colitis, chronic constipation, chronic diarrhea, dysentery, peptic ulcer disease (e.g., stomach ulcer or duodenal ulcer), gastritis, gastroenteritis (gastrointestinal infection), gastroesophageal reflux disease (GERD), acid reflux, functional dyspepsia, non-ulcer dyspepsia, gastroparesis (slow gastric emptying), Barrett's esophagus, achalasia, non-achalasia esophageal motility disorders, anorexia nervosa, bulimia nervosa, orthorexia nervosa, avoidant restrictive food intake disorder (ARFID), diabulimia, other specified feeding and eating disorders (OSFED; e.g., atypical anorexia), intestinal cancer, colorectal cancer, appendicitis, diverticulitis, lactose intolerance, short bowel syndrome, abdominal adhesions, Whipple's disease, Pearson marrow-pancreas syndrome, Alagille syndrome, and Addison's anemia (pernicious anemia). In some embodiments, a subject has previously undergone one or more surgical procedures associated with impaired fat absorption selected from, but not limited to: gastric resection, gastrectomy, large bowel resection (e.g., partial colectomy or total colectomy), small bowel resection, proctocolectomy, pancreatectomy, pancreaticoduodenectomy (Whipple procedure), cholecystectomy, and solid organ transplantation. In some embodiments, a subject is recovering from an injury (e.g., an organ injury, a muscle injury, or a bone injury). In some embodiments, a subject is suspected of having or known to have impaired fat absorption resulting from infection of the gastrointestinal tract by one or more pathogens, such as, but not limited to: a bacterium (e.g., Clostridium difficile, Escherichia coli, Salmonella spp., Shigella spp., or Campylobacter spp.), a virus (e.g., a norovirus, a rotavirus, an adenovirus, or an astrovirus), or a parasite (e.g., Giardia duodenalis, Entamoeba histolytica, Cyclospora cayetanenensis, Cryptosporidium spp., Strongyloides stercoralis (threadworm), Necator americanus (hookworm), Ancylostoma duodenale (Old World hookworm), Ascaris lumbricoides (roundworm), Trichiuris trichiuria (whipworm), or Eucestoda spp. (tapeworm)).

In some embodiments, a subject is suspected of having or is known to have transiently impaired fat absorption resulting from (e.g., as a side effect from) a treatment that was previously administered to the subject, such as, but not limited to: an antibiotic, an anti-bacterial agent, an anti-viral agent, an anti-fungal agent, an antiparasitic agent, an anti-inflammatory agent, an immunostimulant, an immunosuppressant, an antiproliferative agent (an antimetabolite), an antidepressant, a steroid, an opiate, an opioid, radiotherapy, or a chemotherapeutic or radiopharmaceutical agent.

In some embodiments, a subject has or is suspected to have diminished fat consumption and/or fat absorption relative to their age group, as measured by caloric intake (kcal/day), caloric intake from fat, height-for-age, weight-for-age, body mass index-for-age, coefficient of fat absorption (CFA), fat mass (FM), fat free mass (FFM), stool fat, plasma fatty acid concentration, and serum fat-soluble vitamin concentration each. Fat consumption and/or fat absorption of a subject compared to their age group may be measured by any means readily known to those skilled in the relevant art and may be expressed, for example, as a percentile or a statistical score (e.g., Z-score). In some embodiments, a subject exhibits a failure to thrive, growth faltering, or unintentional or unexplained weight loss.

In some embodiments, the improvement of dietary fat absorption in the subject upon administration of a matrix comprising LPC or a formulation thereof is positively correlated with the severity of fat malabsorption in the subject.

In some embodiments, a matrix comprising LPC or a formulation thereof is administered prior to, with (e.g., concurrently and/or in combination with), or following administration of a treatment for a disorder (e.g., a disorder associated with impaired fat absorption). In some embodiments, a treatment for a disorder (e.g., a disorder associated with impaired fat absorption) is administered to the subject enterally (i.e., consumed orally or by feeding tube). In some embodiments, a treatment for a disorder (e.g., a disorder associated with impaired fat absorption) is administered to the subject by another route (e.g., topically).

In some embodiments, a matrix comprising LPC or a formulation thereof is administered prior to, with (e.g., concurrently and/or in combination with), or following administration of a treatment for EPI. A treatment for EPI may comprise any therapeutic readily known in the art to be effective for treating EPI such as, but not limited to, pancreatic enzyme replacement therapy (PERT). PERT therapeutics comprising pancreatic lipase, amylase, and pancreatic proteases are readily available from commercial sources.

In some embodiments, a matrix comprising LPC or a formulation thereof is administered prior to, with (e.g., concurrently and/or in combination with), or following administration of a treatment for pancreatic cancer. A treatment for pancreatic cancer may comprise any therapeutic readily known in the art to be effective for treating pancreatic cancer such as, but not limited to, gemcitabine, 5-fluorouracil, oxaliplatin, cisplatin, capecitabine, irinotecan, and taxanes (e.g., paclitaxel, albumin-bound paclitaxel, docetaxel).

In some embodiments, a matrix comprising LPC or a formulation thereof is administered prior to, with (e.g., concurrently and/or in combination with), or following administration of a treatment for cystic fibrosis. In some embodiments, the treatment for cystic fibrosis comprises a therapeutic that is a cystic fibrosis transmembrane conductance regulator (CFTR) corrector, a CFTR potentiator, or a combination thereof. A CFTR corrector may be any therapeutic compound known in the art to facilitate protein folding of CFTR channels and trafficking of assembled CFTR channels to the apical surface of cells lining the digestive tract, such as lumacaftor (VX-809), tezacaftor (VX-661) or elexacaftor (VX-445). A CFTR potentiator may be any therapeutic compound known in the art to facilitate function of assembled CFTR channels at the apical surface of cells lining the digestive tract, such as ivacaftor (VX-770). Commercially available combinations of CFTR correctors and potentiators known in the art for administration to subjects with cystic fibrosis include Orkambi® (lumacaftor/ivacaftor), Symdeko® (tezacaftor/ivacaftor), and Trikafta® (tezacaftor/elexacaftor/ivacaftor). Additional details for commercially available CFTR correctors and potentiators that may be administered as a treatment for cystic fibrosis may be found, for instance, in Middleton P G, et al. “Elexacaftor-Tezacaftor-Ivacaftor for Cystic Fibrosis with a Single Phe508del Allele.” N Engl J Med. 2019 Nov. 7; 381(19):1809-1819; and Wainwright C E, et al. “Lumacaftor-Ivacaftor in Patients with Cystic Fibrosis Homozygous for Phe508del CFTR.” N Engl J Med. 2015 Jul. 16; 373(3):220-31. In other embodiments, the treatment for cystic fibrosis is treatment known in the art that does not comprise a CFTR corrector or CFTR potentiator such as, but not limited to, a treatment comprising a proton pump inhibitor, an H2 blocker, ursodeoxycholic acid, hypertonic saline, a mucolytic (e.g., dornase alfa, i.e. Pulmozyme®), an antibiotic (e.g., tobramycin, aztreonam, colistin, azithromycin), a corticosteroid, a leukotriene modifier, a beta agonist, or an anticholinergic, or a treatment comprising any combination of therapeutic agents thereof. In some embodiments, such a treatment for cystic fibrosis further comprises a CFTR corrector or CFTR potentiator.

In some embodiments, a matrix comprising LPC or a formulation thereof that is administered to a subject is formulated to comprise at least 0.5%, at least 1%, at least 2%, at least 3% at least 4%, at least 5%, at least 6%, at least 7%, at least 8%, at least 9% or at least 10% LPC by weight, but less than 20% LPC by weight. In some embodiments, a matrix comprising LPC or a formulation thereof is at least 1% but less than 5% LPC by weight.

In some embodiments, a matrix comprising LPC or a formulation thereof that is administered to a subject is formulated as a solid (e.g., a dry powder), as a semi-solid (e.g., as a paste), or as a liquid. In some embodiments, a matrix comprising LPC or a formulation thereof that is administered to a subject is formulated as a dry powder. In embodiments where a formulation comprising a matrix comprising LPC is administered to a subject is in the form of a dry powder that is administered in the presence of a food or beverage, the dry powder may be combined (e.g., mixed) with the food or beverage prior to administration to (e.g., consumption by) the subject.

In some embodiments, the method comprises administering to a subject at least 0.5 grams, at least 1 gram, at least 2 grams, at least 3 grams, at least 4 grams, at least 5 grams, at least 6 grams, at least 7 grams, at least 8 grams, at least 9 grams, at least 10 grams, at least 15 grams, at least 20 grams, or at least 25 grams of a matrix comprising LPC or a formulation thereof. In some embodiments, between 0.5 grams and 50 grams of a matrix comprising LPC or a formulation thereof are administered to a subject.

In some embodiments, a matrix comprising LPC or a formulation thereof is administered to a subject at least once per day or may be administered intermittently over a week (7 days). In some embodiments, a matrix comprising LPC or a formulation thereof is administered to a subject multiple times per day (i.e., at least twice per day). In some embodiments, a matrix comprising LPC or a formulation thereof is administered to a subject multiple times per day as needed to achieve a dose goal, desired weight gain, or other therapeutic goal(s) (e.g., less abdominal pain, fewer bowel movements). In some embodiments, a matrix comprising LPC or a formulation thereof is administered to a subject more than three times per day. In some embodiments, the matrix comprising LPC or a formulation thereof is administered to a subject in combination with a food or beverage, which may include mixing the matrix comprising LPC or a formulation thereof with one or more parts of the food or beverage.

In some embodiments, administration of a matrix comprising LPC or a formulation thereof to a subject improves one or more parameters in the subject, such as those pertaining to growth and/or fat absorption. In some embodiments, administration of a matrix comprising LPC or a formulation thereof to a subject improves one or more of the following parameters in the subject: caloric intake (kcal/day), caloric intake from fat, height-for-age Z score (HAZ), weight-for-age Z score (WAZ), body mass index-for-age Z score (BMIZ), coefficient of fat absorption (CFA), fat mass (FM), fat free mass (FFM), stool fat, plasma fatty acid concentration, and serum fat-soluble vitamin concentration. In embodiments where the subject is a subject suspected of having or known to have cystic fibrosis, administration of a matrix comprising LPC or a formulation thereof improves a parameter of respiratory function in the subject, e.g., forced expiratory volume at 1 second percent predicted value (FEV₁).

In embodiments where the subject is a child (e.g., a human child), administration of a matrix comprising LPC or a formulation thereof to the subject is followed by a normalization of growth (e.g., weight, size) in the subject, as determined by measuring one or more parameters (e.g., height-for-age Z score (HAZ), weight-for-age Z score (WAZ), body mass index-for-age Z score (BMIZ)). In embodiments where the subject is an adult (e.g., a human adult) or elder (e.g., an elderly human), administration of a matrix comprising LPC or a formulation thereof to the subject is followed by an improvement in the weight status of the subject, as determined by measuring one or more parameters (e.g., weight-for-age Z score (WAZ), body mass index-for-age Z score (BMIZ)). In some embodiments, administration of a matrix comprising LPC or a formulation thereof to an adult (e.g., a human adult) or elder (e.g., an elderly human) improves the management of one or more diseases or disorders in the subject.

In some embodiments, administration of a matrix comprising LPC or a formulation thereof to a subject further comprises testing if the administration improves fat absorption in the subject. In some embodiments, testing whether the administration improves fat absorption in the subject comprises measuring one or more of the following parameters in the subject and comparing to a corresponding measurement prior to the administration: caloric intake (kcal/day), caloric intake from fat, height-for-age Z score (HAZ), weight-for-age Z score (WAZ), body mass index-for-age Z score (BMIZ), coefficient of fat absorption (CFA), fat mass (FM), fat free mass (FFM), and stool fat, techniques for the measurement of which are widely known in the relevant art. In some embodiments, testing whether the administration improves fat absorption in the subject comprises collecting a biological sample from the subject (e.g., a biological fluid, e.g., a blood sample, a serum sample, a plasma sample), measuring the level of one or more nutritional status biomarkers in the biological sample, and comparing the level of the one or more nutritional status biomarkers to the level of the one or more nutritional status biomarkers measured in a corresponding biological sample collected prior to the administration. A “nutritional status biomarker” may be any of the following without limitation: a fat for calories (caloric fat, e.g., a saturated fatty acid, a monounsaturated fatty acid, or a polyunsaturated fatty acid), an essential fatty acid (i.e., a fatty acid that cannot be synthesized by cells of the subject, e.g., α-linolenic acid, linolenic acid, pentadecanoic acid), or a serum fat soluble vitamin (e.g., vitamin A, vitamin D, vitamin E, or vitamin K). In some embodiments, administration of a matrix comprising LPC or a formulation thereof to a subject is determined to improve fat absorption in the subject if the level of one or more nutritional status biomarkers measured in a biological sample collected from the subject after administration is increased (e.g., significantly increased) compared to the level of the one or more nutritional status biomarkers in a corresponding biological sample collected prior to the administration.

All publications, patents, patent applications, publication, and database entries (e.g., sequence database entries) mentioned herein, e.g., in the Background, Summary, Detailed Description, Examples, and/or References sections, are hereby incorporated by reference in their entirety as if each individual publication, patent, patent application, publication, and database entry was specifically and individually incorporated herein by reference. In case of conflict, the present application, including any definitions herein, will control.

Certain embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Example 1: Increase in Typical Dietary Fat when Consumed with a New Lysophosphatidylcholine Rich Nutritional Therapy

### Background

Encala™ is an oral medical nutrition therapy that provides highly absorbable fat calories, is independent of lipase enzymes and bile acids for digestion and absorption and has been shown to improve weight, growth, choline and essential fatty acid status in people with cystic fibrosis (CF). Encala is the clinically available version of LXS used in our CF and pancreatic insufficiency (PI) randomized, double-blind, placebo controlled clinical trial (RCT). Encala is a lysophosphatidylcholine (LPC) rich structured lipid compound that forms a micelle-like structure in the small intestine and significantly increases fat absorption in people with disease as well as in healthy people. MDR3 is a well characterized gut and biliary mucosal cell membrane efflux pump that transports intracellular LPC and phosphatidylcholine back into gut lumen. This analysis was to determine if the intestine luminal and mucosal cell environment created by mucosal cell to gut MDR3-recycled LPC from Encala altered fat absorption of accompanying foods.

### Methods

Plasma palmitic acid (C16:0) and other fatty acid (FA) concentrations were determined by standard methods (Mayo Clinical Laboratory) in all subjects and the subset of subjects with severe baseline fat malabsorption (Coefficient of fat absorption [% CFA<median {88%}]). Baseline and 3-month concentrations were compared by randomized treatment group (Encala vs placebo [PLA]). C16:0 was selected as the indicator dietary fatty acid as it is a frequently consumed fatty acid and a minor component of Encala fat. Caloric and fat contents of Encala and placebo were the same. Participants were 5-18 yrs old with CF and PI who participated in the RCT, were on standard CF medications and other therapy, and were enrolled from 10 CF Centers that managed all clinical care.

### Results

TABLE 1

| | | All Subjects | | | | | Subjects with CFA <88% | | | | |
|---|---|---|---|---|---|---|---|---|---|---|---|
| | | | | | % | | | | | % | |
| | | n | Baseline | 3-months | Change | P value | n | Baseline | 3-months | Change | P value |
| C16:0, | PLA | 56 | 2267 | 2275 | 0 | — | 14 | 2304 | 2190 | −5 | — |
| nmol/L | Encala | 51 | 2064 | 2560 | 24 | 0.001 | 19 | 2038 | 2498 | 23 | 0.007 |
| Total FA, | PLA | 56 | 9.16 | 9.12 | 0 | — | 14 | 9.25 | 8.62 | −7 | — |
| mmol/L | Encala | 51 | 8.56 | 10.14 | 18 | 0.001 | 19 | 8.43 | 10.15 | 20 | 0.005 |

As shown in Table 1, after three months of therapy, plasma C16:0 concentration increased 24% (p=0.001) in all subjects on Encala and 23% (p=0.007) in the subjects on Encala with more severe fat malabsorption at baseline. There were no changes in C16:0 or other FA in subjects on PLA. A similar pattern of change in plasma Total FA concentrations for the Encala and PLA groups was demonstrated (Table 1). Plasma total monounsaturated and polyunsaturated fatty acid concentrations increased (p<0.05) with Encala and no significant change in placebo.

### CONCLUSION

Plasma C16:0 and other lipids concentration increased and indicate that LPC from Encala and the action of MDR3-recycled LPC create an intra-luminal environment results in increased accompanying dietary fat absorption. This second mechanism of action for Encala therapy results in increased calories to the patient, in addition to calories provided directly by Encala.

### REFERENCES

Stallings, et al., PLoS One. 2020 May 8; 15(5):e0232685.
Stallings, et al., J Pediatr Gastroenterol Nutr. 2016 December; 63(6):676-680.
Schall, et al., J Pediatr Gastroenterol Nutr. 2016 April; 62(4):618-26.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

Where websites are provided, URL addresses are provided as non-browser-executable codes, with periods of the respective web address in parentheses. The actual web addresses do not contain the parentheses.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the disclosure, can be excluded from any one or more claims. For purposes of brevity, all of the embodiments in which one or more elements, features, purposes, or aspects is excluded are not set forth explicitly herein.

What is claimed is:

1. A method of increasing dietary fat absorption comprising administering an effective amount of a matrix comprising lysophosphatidylcholine (LPC) in combination with a food or beverage to a subject having impaired dietary fat absorption.

2. The method of claim 1, wherein dietary fat absorption in the subject is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

3. The method of claim 1 or claim 2, wherein the subject absorbs dietary fat from the matrix comprising LPC.

4. The method of claim 1 or claim 2, wherein the subject absorbs dietary fat from the food or beverage.

5. The method of claim 4, wherein the subject absorbs dietary fat from the food or beverage and not from the matrix comprising LPC.

6. The method of claim 4 or 5, wherein the subject absorbs dietary fat from the food or beverage through recycling of LPC in the matrix from the circulatory system of the subject to the digestive tract.

7. The method of any one of claims 1-6, wherein the administration increases the absorption of monounsaturated, polyunsaturated, and/or saturated fatty acids in the subject.

8. The method of claim 7, wherein the fatty acids comprise one or more of linoleic acid, α-linolenic acid, γ-linolenic acid, pentadecanoic acid, arachidonic acid, eicosapentaenoic acid, docosapentaenoic acid, and docosahexaenoic acid.

9. The method of any one of claims 1-8, wherein dietary fat absorption in the subject is increased independently of lipase activity and/or bile acids in the subject.

10. The method of any one of claims 1-9, wherein the administration increases the absorption of fat-soluble vitamins and/or anti-oxidants in the subject.

11. The method of claim 10, wherein the fat-soluble vitamins comprise one or more of vitamin A, vitamin D, vitamin E, and vitamin K.

12. The method of any one of claims 1-11, wherein the subject is a human suspected of having or known to have a disorder associated with impaired fat absorption.

13. The method of claim 12, wherein the subject is a human neonate, a human infant, a human child, a human adult, or an elderly human.

14. The method of claim 12, wherein the subject is an elderly human with frailty syndrome.

15. The method of any one of claims 1-11, wherein the subject is a non-human animal, a domestic animal, or a companion animal suspected of having or known to have a disorder associated with impaired fat absorption.

16. The method of any one of claims 12-15, wherein the disorder is a disease associated with exocrine pancreatic insufficiency (EPI).

17. The method of claim 16, wherein the disorder is selected from the group consisting of cystic fibrosis, chronic pancreatitis, pancreatic cancer, type 1 diabetes mellitus, type 2 diabetes mellitus, type 3c diabetes mellitus, protein-calorie malnutrition, age-related lipase insufficiency, Schwachman-Diamond syndrome, Johanson-Blizzard syndrome, Zollinger-Ellison syndrome, and lipase/co-lipase enzyme deficiency.

18. The method of any one of claims 12-15, wherein the disorder is a disease associated with bile acid insufficiency.

19. The method of claim 18, wherein the disorder is selected from the group consisting of cholestasis, cirrhosis, biliary atresia, Wilson disease, parental nutrition-associated liver disease, infectious hepatitis, hepatic steatosis with obesity, bile acid metabolism disorders, bilirubin metabolism disorders, end stage liver disease, and bile acid insufficiency associated with liver transplant.

20. The method of any one of claims 12-15, wherein the disorder is selected from the group consisting of malabsorption syndrome, alcoholic pancreatitis, pancreatic duct obstruction, hereditary hemochromatosis, liver disease, liver cancer, bile duct obstruction, bile acid depletion, abetalipoproteinemia, cholecystitis, cholelithiasis, chronic gallbladder disease, gallbladder cancer, inflammatory bowel disease, irritable bowel syndrome, intestinal obstruction, intestinal pseudo-obstruction, intestinal adhesion, Crohn's disease, Celiac disease, ulcerative colitis, infectious colitis, ischemic colitis, radiation colitis, chronic constipation, chronic diarrhea, dysentery, peptic ulcer disease, gastritis, gastroenteritis, gastroesophageal reflux disease, acid reflux, functional dyspepsia, non-ulcer dyspepsia, gastroparesis, Barrett's esophagus, achalasia, non-achalasia esophageal motility disorders, anorexia nervosa, bulimia nervosa, orthorexia nervosa, avoidant restrictive food intake disorder, diabulimia, other specified feeding and eating disorders, intestinal cancer, colorectal cancer, appendicitis, diverticulitis, lactose intolerance, short bowel syndrome, abdominal adhesions, Whipple's disease, Pearson marrow-pancreas syndrome, Alagille syndrome, and Addison's anemia.

21. The method of any one of claims 12-20, wherein the administration is followed by normalization of growth in the subject.

22. The method of any one of claims 12-21, wherein the administration improves weight status and/or management of the disorder associated with impaired fat absorption in the subject.

23. The method of claim 17, wherein the matrix comprising LPC is administered prior to, with, or following administration of a treatment for EPI, a treatment for cystic fibrosis, or a combination thereof.

24. The method of claim 23, wherein the treatment for EPI comprises pancreatic enzyme replacement therapy (PERT).

25. The method of claim 23, wherein the treatment for cystic fibrosis comprises a cystic fibrosis transmembrane conductance regulator (CFTR) corrector, a CFTR potentiator, or a combination thereof.

26. The method of any one of claims 1-11, wherein the subject is a healthy non-human animal, a healthy domestic animal, or a healthy companion animal in which the matrix comprising LPC is administered for the purpose of increasing growth.

27. The method of any one of claims 1-26, wherein administration of the matrix comprising LPC increases the level of one or more nutritional status biomarkers in the subject, wherein the nutritional status biomarkers are selected from at least one fat for calories, at least one essential fatty acid, and/or least one fat soluble vitamin.

28. The method of claim 27, wherein the at least one fat for calories is a fatty acid selected from a saturated fatty acid, a monounsaturated fatty acid, and a polyunsaturated fatty acid, or a combination thereof.

29. The method of claim 27, wherein the at least one essential fatty acid is selected from linolenic acid, α-linolenic acid, and pentadecanoic acid, or a combination thereof.

30. The method of claim 27, wherein the at least one fat soluble vitamin is selected from vitamin A, vitamin D, vitamin E, and vitamin K, or a combination thereof.

31. The method of any one of claims 1-30, wherein the matrix comprising LPC is at least 1% LPC by weight.

32. The method of any one of claims 1-31, wherein the matrix comprising LPC is in the form of a dry powder, a paste, or a liquid.

33. The method of claim 32, wherein the matrix comprising LPC is in the form of a dry powder that is mixed with the food or beverage.

34. The method of any one of claims 1-33, wherein 0.5-50 g of the matrix comprising LPC are administered to the subject.

35. The method of any one of claims 1-34, wherein the matrix comprising LPC is administered at least once per day and/or at least once per week.

36. The method of claim 35, wherein the matrix comprising LPC is administered at least twice per day.

37. A method of increasing fat absorption from the digestive tract comprising administering an effective amount of a matrix comprising lysophosphatidylcholine (LPC) to a subject having impaired dietary fat absorption, whereby absorption of fat present in the digestive tract is increased.

38. The method of claim 37, wherein absorption of fat present in the digestive tract is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

39. The method of claim 37 or claim 38, wherein the fat absorbed in the digestive tract of the subject is that which is comprised by the matrix comprising LPC.

40. The method of claim 37 or claim 38, wherein the fat absorbed in the digestive tract of the subject is not solely that which is comprised by the matrix comprising LPC.

41. The method of claim 40, wherein the fat absorbed in the digestive tract of the subject is absorbed through recycling of LPC in the matrix from the circulatory system to the digestive tract of the subject.

42. The method of any one of claims 37-41, wherein the administration increases the absorption of monounsaturated, polyunsaturated, and/or saturated fatty acids in the subject.

43. The method of claim 42, wherein the fatty acids comprise one or more of linoleic acid, α-linolenic acid, γ-linolenic acid, pentadecanoic acid, arachidonic acid, eicosapentaenoic acid, docosapentaenoic acid, and docosahexaenoic acid.

44. The method of any one of claims 37-43, wherein dietary fat absorption in the subject is increased independently of lipase activity and/or bile acids in the subject.

45. The method of any one of claims 37-44, wherein the administration increases the absorption of fat-soluble vitamins in the subject.

46. The method of claim 45, wherein the fat-soluble vitamins comprise one or more of vitamin A, vitamin D, vitamin E, and vitamin K.

47. The method of any one of claims 37-46, wherein the subject is a human suspected of having or known to have a disorder associated with impaired fat absorption.

48. The method of claim 47, wherein the subject is a human neonate, a human infant, a human child, a human adult, or an elderly human.

49. The method of claim 47, wherein the subject is an elderly human with frailty syndrome.

50. The method of any one of claims 37-46, wherein the subject is a non-human animal, a domestic animal, or a companion animal suspected of having or known to have a disorder associated with impaired fat absorption.

51. The method of any one of claims 47-50, wherein the disorder is a disease associated with exocrine pancreatic insufficiency (EPI).

52. The method of claim 51, wherein the disorder is selected from the group consisting of cystic fibrosis, chronic pancreatitis, pancreatic cancer, type 1 diabetes mellitus, type 2 diabetes mellitus, type 3c diabetes mellitus, protein-calorie malnutrition, age-related lipase insufficiency, Schwachman-Diamond syndrome, Johanson-Blizzard syndrome, Zollinger-Ellison syndrome, and lipase/co-lipase enzyme deficiency.

53. The method of any one of claims 47-50, wherein the disorder is a disease associated with bile acid insufficiency.

54. The method of claim 53, wherein the disorder is selected from the group consisting of cholestasis, cirrhosis, biliary atresia, Wilson disease, parental nutrition-associated liver disease, infectious hepatitis, hepatic steatosis with obesity, bile acid metabolism disorders, bilirubin metabolism disorders, end stage liver disease, and bile acid insufficiency associated with liver transplant.

55. The method of any one of claims 47-50, wherein the disorder is selected from the group consisting of malabsorption syndrome, alcoholic pancreatitis, pancreatic duct obstruction, hereditary hemochromatosis, liver disease, liver cancer, bile duct obstruction, bile acid depletion, abetalipoproteinemia, cholecystitis, cholelithiasis, chronic gallbladder disease, gallbladder cancer, inflammatory bowel disease, irritable bowel syndrome, intestinal obstruction, intestinal pseudo-obstruction, intestinal adhesion, Crohn's disease, Celiac disease, ulcerative colitis, infectious colitis, ischemic colitis, radiation colitis, chronic constipation, chronic diarrhea, dysentery, peptic ulcer disease, gastritis, gastroenteritis, gastroesophageal reflux disease, acid reflux, functional dyspepsia, non-ulcer dyspepsia, gastroparesis, Barrett's esophagus, achalasia, non-achalasia esophageal motility disorders, anorexia nervosa, bulimia nervosa, orthorexia nervosa, avoidant restrictive food intake disorder, diabulimia, other specified feeding and eating disorders, intestinal cancer, colorectal cancer, appendicitis, diverticulitis, lactose intolerance, short bowel syndrome, abdominal adhesions, Whipple's disease, Pearson marrow-pancreas syndrome, Alagille syndrome, and Addison's anemia.

56. The method of any one of claims 47-55, wherein the administration is followed by normalization of growth in the subject.

57. The method of any one of claims 47-56, wherein the administration improves weight status and/or management of the disorder associated with impaired fat absorption in the subject.

58. The method of claim 52, wherein the matrix comprising LPC is administered prior to, with, or following administration of a treatment for EPI, a treatment for cystic fibrosis, or a combination thereof.

59. The method of claim 58, wherein the treatment for EPI comprises pancreatic enzyme replacement therapy (PERT).

60. The method of claim 58, wherein the treatment for cystic fibrosis comprises a cystic fibrosis transmembrane conductance regulator (CFTR) corrector, a CFTR potentiator, or a combination thereof.

61. The method of any one of claims 37-46, wherein the subject is a healthy domestic animal or a companion animal in which the matrix comprising LPC is administered for the purpose of increasing growth.

62. The method of any one of claims 37-61, wherein administration of the matrix comprising LPC increases the level of one or more nutritional status biomarkers in the subject, wherein the nutritional status biomarkers are selected from at least one fat for calories, at least one essential fatty acid, and/or least one fat soluble vitamin.

63. The method of claim 62, wherein the at least one fat for calories is a fatty acid selected from a saturated fatty acid, a monounsaturated fatty acid, and a polyunsaturated fatty acid, or a combination thereof.

64. The method of claim 62, wherein the at least one essential fatty acid is selected from linolenic acid, α-linolenic acid, and pentadecanoic acid, or a combination thereof.

65. The method of claim 62, wherein the at least one fat soluble vitamin is selected from vitamin A, vitamin D, vitamin E, and vitamin K, or a combination thereof.

66. The method of any one of claims 37-65, wherein the matrix comprising LPC is at least 1% LPC by weight.

67. The method of any one of claims 37-66, wherein the matrix comprising LPC is in the form of a dry powder, a paste, or a liquid.

68. The method of claim 67, wherein the matrix comprising LPC is in the form of a dry powder.

69. The method of any one of claims 37-68, wherein 0.5-50 g of the matrix comprising LPC are administered to the subject.

70. The method of any one of claims 37-69, wherein the matrix comprising LPC is administered at least once per day and/or at least once per week.

71. The method of claim 70, wherein the matrix comprising LPC is administered at least twice per day.

72. A method of increasing the concentration of fatty acids in plasma of a subject having impaired dietary fat absorption, comprising administering to the subject a source of fat and an effective amount of a matrix comprising lysophosphatidylcholine (LPC), whereby the subject absorbs fatty acids from the source of fat.

73. The method of claim 72, wherein the source of fat is a food or beverage.

74. The method of claim 72 or claim 73, wherein fatty acids from the source of fat are absorbed in the digestive tract of the subject through recycling of LPC in the matrix from the circulatory system to the digestive tract of the subject.

75. The method of any one of claims 72-74, wherein the fatty acids comprise monounsaturated, polyunsaturated, and/or saturated fatty acids.

76. The method of any one of claims 72-75, wherein the fatty acids comprise one or more of linoleic acid, α-linolenic acid, γ-linolenic acid, pentadecanoic acid, arachidonic acid, eicosapentaenoic acid, docosapentaenoic acid, and docosahexaenoic acid.

77. The method of any one of claims 72-76, wherein the concentration of fatty acids in plasma of the subject is increased by at least 10%, increased by at least 20%, increased by at least 30%, increased by at least 40%, increased by at least 50%, increased by at least 60%, increased by at least 70%, increased by at least 80%, increased by at least 90%, increased by at least 2-fold, increased by at least 3-fold, increased by at least 4-fold, increased by at least 5-fold, or increased by at least 10-fold.

78. The method of any one of claims 72-77, wherein fatty acid absorption in the subject is increased independently of lipase activity and/or bile acids in the subject.

79. The method of any one of claims 72-78, wherein the administration further increases the concentration of fat-soluble vitamins in serum of the subject.

80. The method of claim 79, wherein the fat-soluble vitamins comprise one or more of vitamin A, vitamin D, vitamin E, and vitamin K.

81. The method of any one of claims 72-80, wherein the subject is a human suspected of having or known to have a disorder associated with impaired fat absorption.

82. The method of claim 81, wherein the subject is a human neonate, a human infant, a human child, a human adult, or an elderly human.

83. The method of claim 81, wherein the subject is an elderly human with frailty syndrome.

84. The method of any one of claims 72-80, wherein the subject is a non-human animal, a domestic animal, or a companion animal suspected of having or known to have a disorder associated with impaired fat absorption.

85. The method of any one of claims 81-84, wherein the disorder is a disease associated with exocrine pancreatic insufficiency (EPI).

86. The method of claim 85, wherein the disorder is selected from the group consisting of cystic fibrosis, chronic pancreatitis, pancreatic cancer, type 1 diabetes mellitus, type 2 diabetes mellitus, type 3c diabetes mellitus, protein-calorie malnutrition, age-related lipase insufficiency, Schwachman-Diamond syndrome, Johanson-Blizzard syndrome, Zollinger-Ellison syndrome, and lipase/co-lipase enzyme deficiency.

87. The method of any one of claims 81-84, wherein the disorder is a disease associated with bile acid insufficiency.

88. The method of claim 87, wherein the disorder is selected from the group consisting of cholestasis, cirrhosis, biliary atresia, Wilson disease, parental nutrition-associated liver disease, infectious hepatitis, hepatic steatosis with obesity, bile acid metabolism disorders, bilirubin metabolism disorders, end stage liver disease, and bile acid insufficiency associated with liver transplant.

89. The method of any one of claims 81-84, wherein the disorder is selected from the group consisting of malabsorption syndrome, alcoholic pancreatitis, pancreatic duct obstruction, hereditary hemochromatosis, liver disease, liver cancer, bile duct obstruction, bile acid depletion, abetalipoproteinemia, cholecystitis, cholelithiasis, chronic gallbladder disease, gallbladder cancer, inflammatory bowel disease, irritable bowel syndrome, intestinal obstruction, intestinal pseudo-obstruction, intestinal adhesion, Crohn's disease, Celiac disease, ulcerative colitis, infectious colitis, ischemic colitis, radiation colitis, chronic constipation, chronic diarrhea, dysentery, peptic ulcer disease, gastritis, gastroenteritis, gastroesophageal reflux disease, acid reflux, functional dyspepsia, non-ulcer dyspepsia, gastroparesis, Barrett's esophagus, achalasia, non-achalasia esophageal motility disorders, anorexia nervosa, bulimia nervosa, orthorexia nervosa, avoidant restrictive food intake disorder, diabulimia, other specified feeding and eating disorders, intestinal cancer, colorectal cancer, appendicitis, diverticulitis, lactose intolerance, short bowel syndrome, abdominal adhesions, Whipple's disease, Pearson marrow-pancreas syndrome, Alagille syndrome, and Addison's anemia.

90. The method of any one of claims 81-88, wherein the administration is followed by normalization of growth in the subject.

91. The method of any one of claims 81-90, wherein the administration improves weight status and/or management of the disorder associated with impaired fat absorption in the subject.

92. The method of claim 86, wherein the matrix comprising LPC is administered prior to, with, or following administration of a treatment for EPI, a treatment for cystic fibrosis, or a combination thereof.

93. The method of claim 92, wherein the treatment for EPI comprises pancreatic enzyme replacement therapy (PERT).

94. The method of 92, wherein the treatment for cystic fibrosis comprises a cystic fibrosis transmembrane conductance regulator (CFTR) corrector, a CFTR potentiator, or a combination thereof.

95. The method of any one of claims 72-80, wherein the subject is a healthy domestic animal or a companion animal in which the matrix comprising LPC is administered for the purpose of increasing growth.

96. The method of any one of claims 72-95, wherein administration of the matrix comprising LPC increases the level of one or more nutritional status biomarkers in the subject, wherein the nutritional status biomarkers are selected from at least one fat for calories, at least one essential fatty acid, and/or least one fat soluble vitamin.

97. The method of claim 96, wherein the at least one fat for calories is a fatty acid selected from a saturated fatty acid, a monounsaturated fatty acid, and a polyunsaturated fatty acid, or a combination thereof.

98. The method of claim 96, wherein the at least one essential fatty acid is selected from linolenic acid, α-linolenic acid, and pentadecanoic acid, or a combination thereof.

99. The method of claim 96, wherein the at least one fat soluble vitamin is selected from vitamin A, vitamin D, vitamin E, and vitamin K, or a combination thereof.

100. The method of any one of claims 72-99, wherein the matrix comprising LPC is at least 1% LPC by weight.

101. The method of any one of claims 72-100, wherein the matrix comprising LPC is in the form of a dry powder, a paste, or a liquid.

102. The method of claim 101, wherein the matrix comprising LPC is in the form of a dry powder.

103. The method of any one of claims 72-102, wherein 0.5-50 g of the matrix comprising LPC are administered to the subject.

104. The method of any one of claims 72-103, wherein the matrix comprising LPC is administered at least once per day and/or at least once per week.

105. The method of claim 104, wherein the matrix comprising LPC is administered at least twice per week.
